(19) United States
(12) Patent Application Publication
(10) Pub. No.: US 2011/0200536 A1
(43) Pub. Date: Aug. 18, 2011

(54) CHELATORS, PARAMAGNETIC CHELATES THEREOF AND THEIR USE AS CONTRAST AGENTS IN MAGNETIC RESONANCE IMAGING (MRI)

(76) Inventors: Harry John Wadsworth, Amersham (GB); Ian Martin Newington, Amersham (GB); Dennis O'shea, Amersham (GB)

(21) Appl. No.: 12/672,117

(22) PCT Filed: Aug. 12, 2008
(86) PCT No.: PCT/EP2008/060572

§ 371 Date: May 3, 2011

(30) Foreign Application Priority Data
Aug. 13, 2007 (NO) 20074165

(51) Int. Cl. A61K 49/10; C07D 407/14; C07D 401/14; C07D 403/14; C07K 2/00; C07K 16/00; A61P 43/00; C08F 8/30; C08F 138/02; B82Y 5/00

(52) U.S. Cl. 424/9.361; 549/415; 546/261; 546/2; 548/255; 530/300; 530/350; 530/391.1; 525/153; 977/773

(57) ABSTRACT

The present invention relates to chelators, in particular to chelators which are capable of forming complexes, i.e. paramagnetic chelates, with paramagnetic metal ions. The invention also relates to said paramagnetic chelates, said paramagnetic chelates linked to other molecules and their use as contrast agents in magnetic resonance imaging (MRI).

The present invention relates to chelators, in particular to chelators which are capable of forming complexes, i.e. paramagnetic chelates, with paramagnetic metal ions. The invention also relates to said paramagnetic chelates, said paramagnetic chelates linked to other molecules and their use as contrast agents in magnetic resonance imaging (MRI).

MRI is a medical imaging technique in which areas of the body are visualised via the nuclei of selected atoms, especially hydrogen nuclei. The MRI signal depends upon the environment surrounding the visualised nuclei and their longitudinal and transverse relaxation times, T₁ and T₂. Thus, in the case when the visualised nucleus is a proton, the MRI signal intensity will depend upon factors such as proton density and the chemical environment of the protons. Contrast agents are often used in MRI in order to improve the imaging contrast. They work by effecting the T₁, T₂ and/or T₂* relaxation time and thereby influence the contrast in the images.

Several types of contrast agents have been used in MRI. Blood pool MR contrast agents, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

Water-soluble paramagnetic chelates, i.e. complexes of a chelator and a paramagnetic metal ion—for instance gadolinium chelates like Omniscan™ (GE Healthcare)—are widely used MR contrast agents. Because of their low molecular weight they rapidly distribute into the extracellular space (i.e. the blood and the interstitium) when administered into the vasculature. They are also cleared relatively rapidly from the body.

The problem with the in vivo use of paramagnetic metal ions in a MRI contrast agent is their toxicity and therefore they are provided as complexes with chelators which are more stable and less toxic.

For a paramagnetic chelate to be useful as a contrast agent in MRI, it is necessary for it to have certain properties. Firstly, it must have high stability because it is important that the complex does not break down in situ and release toxic paramagnetic metal ions into the body.

Secondly, in order for it to be a potent MRI contrast agent, a paramagnetic chelate must have high relaxivity. The relaxivity of a MRI contrast agent refers to the amount of increase in signal intensity (i.e. decrease in T₁) that occurs per mole of metal ions. Relaxivity is dependent upon the water exchange kinetics of the paramagnetic chelate.

The solubility of the paramagnetic chelate in water is also an important factor when they are used as contrast agents for MRI because they are administered to patients in relatively large doses. A highly water-soluble paramagnetic chelate requires a lower injection volume, is thus easier to administer to a patient and causes less discomfort.

U.S. Pat. No. 5,624,901 and U.S. Pat. No. 5,892,029 both describe a class of chelators based on 1-hydroxy-2-pyridinone and 3-hydroxy-2-pyridinone moieties which have a substituted carbamoyl group adjacent the hydroxyl or oxo groups of the ring. The compounds are said to be useful as actinide sequestering agents for in vivo use because of their ability to form complexes with actinides. However, it does not refer directly to the complexes which are formed or to any possibility of using them as MRI contrast agents.

U.S. Pat. No. 4,666,927 also relates to hydroxypyridinones. The preferred compounds have an oxo group in either the 2- or the 4-position and a hydroxyl group in the 1- or 3-position. The only other ring substituents are alkyl groups and the compounds are said to be useful as agents for the treatment of general iron overload.

US-A-2003/0095922 relates to complexes formed between gadolinium (III) ions and an organic chelator. The chelator is said to be based on a pyridinone, pyrimidinone or pyridazinone ring system. The exemplified pyridinone compounds are all 3-hydroxy-2-pyridinones with a carbamoyl group in the 4-position of the ring. The compounds are said to be useful as MRI contrast agents and to have high solubility and low toxicity.

D. T. Puerta et al, J. Am. Chem. Soc. Vol. 128, No. 7, 2006, 2222-2223 describe gadolinium chelates of 3-hydroxy-4-pyrones, which are high relaxivity MRI contrast agents with moderate solubility.

US-A-2006/0292079 describes bifunctional chelates based on the chelators 3-hydroxypyridine-2-one, and 5-hydroxy-pyrimidin-4-one. The chelates containing gadolinium (III) are used as MRI contrast agents.

The present inventors have developed improved chelators and paramagnetic chelates thereof which can be used as MR contrast agents.

Therefore, in a first aspect of the present invention there is provided a compound of formula (I):

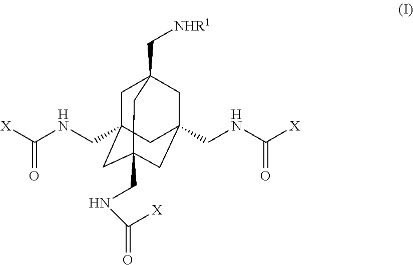

wherein
R¹ is H or C(═O)R², wherein R² is C₁₋C₆-alkyl, C₆₋C₁₅-aryl, C₇₋C₂₂-arylalkyl O—C₁₋C₆-alkyl, O—C₇₋C₂₂-arylalkyl, (CH₂)ₙ—(C₆H₄)—NCS, (CH₂)ₘ—C≡CH or (CH₂)ₘ—N₃, wherein n is 0 to 6 and m is 1 to 6; and
X is a chelator moiety consisting of a 6-membered aromatic or partially saturated ring system containing up to three heteroatoms selected from nitrogen and oxygen and having a hydroxyl group as a first substituent bound to a first atom in said ring system, and a hydroxyl group or an oxygen atom doubly bound to a second atom in said ring system wherein said first and second atom are adjacent atoms and wherein said first and second substituents are in ring positions such that X is capable of forming a complex with a paramagnetic metal ion; and wherein X is optionally substituted by up to three additional substituents, R, where each R is independently a hydrophilic group which renders the compound of formula (I) soluble in aqueous solutions.

The term “chelator” denotes a chemical entity that binds (complexes) a metal ion to form a chelate. If the metal ion is a paramagnetic metal ion, the chemical entity, i.e. complex, formed by said paramagnetic metal ion and said chelator is denoted a paramagnetic chelate. Compounds of formula (I) are chelators since they bind metal ions via their chelator moiety X.

A preferred embodiment of a compound of formula (I) is a compound of formula (II), a paramagnetic chelate, comprising a compound of formula (I) and a paramagnetic metal ion M:

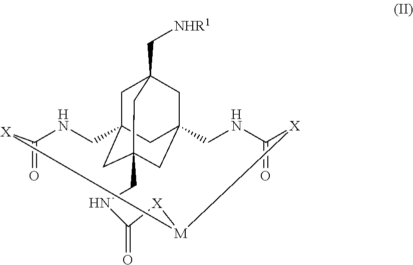

wherein
R¹ and X are as defined above and M is a paramagnetic metal ion.

In the present specification, the term “alkyl” by itself or as part of another substituent refers to a fully saturated straight or branched hydrocarbon chain group having the number of carbon atoms designated. Thus, C₁₋C₆-alkyl means a fully saturated straight or branched hydrocarbon chain group having 1 to 6 carbon atoms and examples of C₁₋C₆-alkyl are methyl, ethyl, n-propyl, isopropyl, n-butyl, isobutyl, t-butyl, n-pentyl, iso-pentyl and n-hexyl.

The term “aryl” by itself or as a part of another substituent refers to an aromatic ring system group consisting of up to three fused or covalently linked rings having the number of carbon atoms designated. Thus, C₆₋C₁₀-aryl refers to an aromatic ring system group consisting of up to three fused or covalently linked rings and having 6 to 10 carbon atoms and examples of C₆₋C₁₀-aryl are phenyl or naphthyl.

The term “arylalkyl” refers to an aryl-substituted alkyl group wherein said aryl and alkyl group are as defined above and wherein said arylalkyl group has the number of carbon atoms designated. Thus, C₇₋C₁₃-arylalkyl refers to an aryl-substituted alkyl group having 7 to 13 carbon atoms and examples of C₇₋C₁₃-arylalkyl are benzyl or phenethyl.

In the present specification the term “paramagnetic metal ion” is a an ion selected from ions of transition and lanthanide metals, i.e. metals of atomic numbers 21 to 29, 42 to 44 or 57 to 71.

Compounds of formula (I) and (II) may exist in either solvated or unsolvated forms and both are encompassed within the scope of the present invention. The present invention also encompasses all solid forms of the compounds, including amorphous and all crystalline forms.

Certain compounds of formula (I) and (II) may exist in different isomeric forms and the present invention is intended to encompass all isomers including enantiomers, diastereoisomers and geometrical isomers as well as racemates.

Compounds of formula (I) and (II) comprise a chelator moiety, i.e. group X. Preferred groups X include groups derived from hydroxypyrones, dihydroxypyridines, hydroxypyrimidones, hydroxypyridones hydroxypyridinones and dihydroxyphenols, any of which may be substituted as described above.

Groups X derived from hydroxypyridones and hydroxypyrimidinones are disclosed in US 2003/0095922.

Groups X derived from hydroxypyridinones which are capable of forming chelates with paramagnetic metal ions are also disclosed in U.S. Pat. No. 4,698,431, U.S. Pat. No. 4,666,927, U.S. Pat. No. 5,624,901 and our own earlier application number PCT/NO2008/000012.

Preferred groups X are of formula (IIIa) to (IIIg)

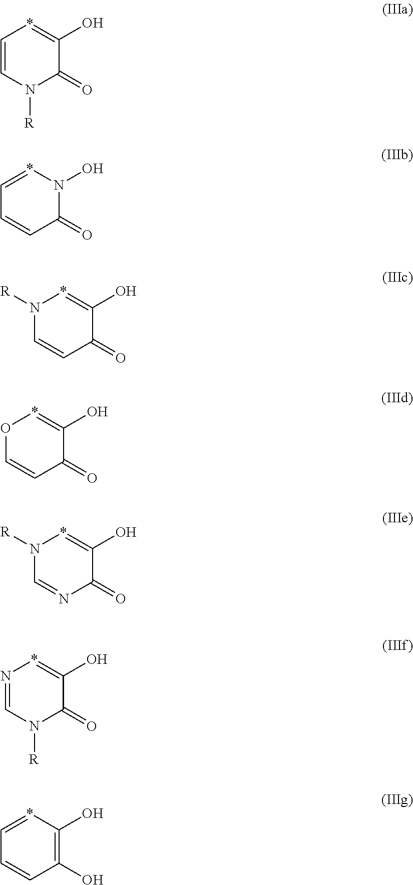

wherein R is as defined in formula (I) and (II) and * indicates the point of attachment of the group X to the remainder of the compound of formula (I) and (II).

In compounds of formula (II) the chelator moieties X form a complex, i.e. paramagnetic chelate, with a paramagnetic metal ion M. In a preferred embodiment, M is a paramagnetic metal ion of Mn, Fe, La, Co, Ni, Eu, Gd, Dy, Tm and Yb, particularly preferred a paramagnetic metal ion of Mn, Fe, La, Eu, Gd and Dy. Most preferably, M is selected from Gd³⁺, Mn²⁺, Fe³⁺, La³⁺, Dy³⁺ and Eu³⁺ with Gd³⁺ being the most preferred paramagnetic metal ion M.

The chelator moieties X in compounds of formula (I) and (II) may be substituted by up to three additional substituents, R, where each R is independently a hydrophilic group which renders the compound of formula (II) soluble in aqueous solutions.

Preferred hydrophilic groups R are groups comprising ester groups, amide groups or amino groups which are optionally further substituted by one or more straight chain or branched C₁-C₁₀-alkyl groups, preferably C₁-C₅-alkyl groups where said alkyl groups also may have one or more CH₂- or CH-moieties replaced by oxygen or nitrogen atoms. The aforementioned preferred hydrophilic groups R may further contain one or more groups selected from hydroxy, amino, oxo, carboxy, amide group, ester group, oxo-substituted sulphur and oxo-substituted phosphorus atoms. The aforementioned straight chain or branched C₁-C₁₀-alkyl groups, preferably C₁-C₅-alkyl groups, preferably contain 1 to 6 hydroxyl groups and more preferably 1 to 3 hydroxyl groups.

Particularly preferred hydrophilic groups R according to the embodiment described above are the following groups R which are attached to a carbon atom in the chelator moiety X and wherein said chelator moiety X is substituted by only 1 of said following groups R. * indicates the point of attachment of the group R to X:

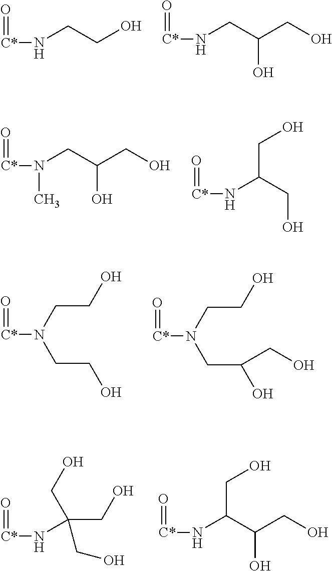

Further preferred hydrophilic groups R are preferably attached to heteroatoms in the chelator moiety X, more preferably attached to nitrogen atoms in the chelator moiety X and such hydrophilic groups R are straight chain or branched C₁-C₁₀-alkyl groups, preferably C₁-C₅-alkyl groups which are substituted by 1 to 6 hydroxyl groups and more preferably by 2 to 5 hydroxyl groups and/or which are substituted by one or more alkyloxy groups, preferably C₁-C₃-alkyloxy groups like methyloxy, ethyloxy and propyloxy groups.

Particularly preferred hydrophilic groups R according to the embodiment described above are the following and * indicates the point of attachment of the group R to X:

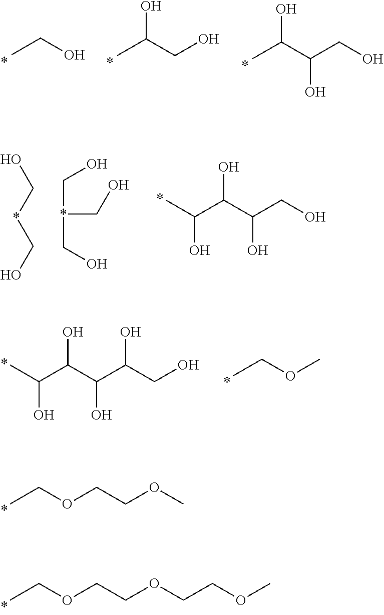

Further preferred hydrophilic groups R are polyethylene glycol groups of up to 3 monomer units.

Further preferred hydrophilic groups R are preferably attached to heteroatoms in the chelator moiety X, more preferably attached to nitrogen atoms in the chelator moiety X and such hydrophilic groups R are groups that comprise up to 3 ethylene oxide units.

Particularly preferred hydrophilic groups R according to the embodiment described above are the following and * indicates the point of attachment of the group R to X:

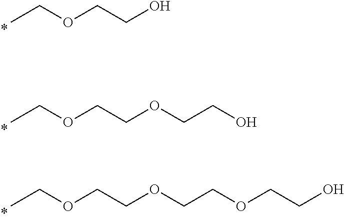

As mentioned before compounds of formula (I) are chelators since they contain chelator moieties X and they can thus be used to chelate metal ions, preferably paramagnetic metal ions. They may or may not be linked via the NHR¹-group to other molecules like natural or synthetic peptides, peptidomimetics, polypeptides, proteins, antibodies, natural or synthetic polymers or dendrimers, nanoparticles or lipophilic compounds.

Compounds of formula (II) can be used as MR contrast agents and may or may not be linked via the NHR¹-group to other molecules such as natural or synthetic peptides, peptidomimetics, polypeptides, proteins or antibodies. By linking compounds of formula (II) to these molecules, targeted MR contrast agents may be obtained if the for instance peptide or protein is a vector which binds to a target like a receptor or cell surface marker. Further, compounds of formula (II) may be linked via the NHR¹-group to polymeric moieties such as natural or synthetic polymers or dendrimers. Such a linking gives compounds of formula (II) a further reduced molecular mobility and therefore increase its relaxivity at high field strengths used in modern MRI scanners. In another embodiment compounds of formula (II) may be linked to lipophilic compounds and the resulting amphiphilic compounds may be dispersed. Such dispersions may be used as MR contrast agent for tumour imaging. In yet another embodiment the compounds of formula (II) may be linked to nanoparticles. Again such a linking gives compounds of formula (II) a further reduced molecular mobility and therefore increases their relaxivity.

Therefore, in a second aspect of the invention there is provided a compound of formula (I) and (II) as defined above linked to another molecule via the NHR¹-group. In a preferred embodiment, said another molecule is a natural or synthetic peptide, a peptidomimetic, a polypeptide, a protein, an antibody, a natural or synthetic polymer, a dendrimer, a nanoparticle or a lipophilic compound.

The term “linked via the NHR¹-group” means that in one embodiment the compounds of formula (I) and (II) are directly linked to another molecule as described above via the NHR¹-group as defined earlier. It is apparent for the skilled person that an NHR¹-group, e.g. an NH₂-group, is a functional group which can be converted to numerous other functional groups by methods known in the art. Thus the term “linked via the NHR¹-group” also includes embodiments wherein the NHR¹-group as defined earlier is first converted into another functional group before the compounds of formula (I) and (II) are then linked to another molecule via said now converted NHR¹-group.

If compounds of formula (I) or (II) are linked to other molecules like natural or synthetic peptides, peptidomimetics, polypeptides, proteins, antibodies, natural or synthetic polymers or dendrimers, R¹ in NHR¹ is preferably H or C(═O)R², wherein R² is (CH₂)ₙ—(C₆H₄)—NCS, (CH₂)ₘ—C≡CH or (CH₂)ₘ—N₃ wherein n is 0 to 6 and m is 1 to 6. In one embodiment, R¹ is H and thus the NHR¹ is a group NH₂ which is a functional group which may be converted to numerous other functional groups by methods known in the art. In order to link a compound of formula (I) or (II) wherein the NHR¹-group is a NH₂-group to another molecule said linking may either be carried out by reacting the NH₂-group of the compound of formula (I) or (II) with a suitable reactive group on said molecule, e.g. reactive groups like acid chlorides or acid anhydrides. Alternatively, the NH₂-group may be converted in a first step to another functional group before compounds of formula (I) or (II) are linked to said other molecule.

If compounds of formula (I) or (II) are linked to other larger molecules like for instance polypeptides, proteins, antibodies or synthetic or natural polymers or dendrimers, R¹ in NHR¹ is preferably C(═O)R², wherein R² is (CH₂)ₙ—(C₆H₄)—NCS, (CH₂)ₘ—C≡CH or (CH₂)ₘ—N₃ wherein n is 0 to 6 and m is 1 to 6. By using compounds of formula (I) or (II) with the aforementioned groups R¹, it is possible to use “click chemistry” (e.g. described by M. Malkoch et al., Macromolecules 38(9), 2005, 3663-3678 or P. Wu et al., Chem. Commun. 46, 2005, 5775-5777). Click chemistry allows linking multiple compounds of formula (I) or (II) to a larger molecule in a very high yielding reaction. Further, the linking reaction can be carried out in conditions that dissolve the reactants such as aqueous conditions.

In a preferred embodiment, R² is as follows and * denotes the attachment point of R² to C of group C(═O)R²:

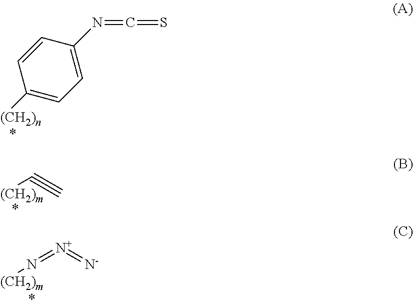

Compounds of formula (I) or (II) wherein R¹ is C(═O)R² and R² is (A), i.e. (CH₂)ₙ—(C₆H₄)—NCS, can be reacted with another (large) molecule comprising amino groups —NH₂ under formation of a thiourea bond (—NH—C(═S)—NH—).

Compounds of formula (I) or (II) wherein R¹ is C(═O)R² and R² is (B), i.e. (CH₂)ₘ—C≡CH, can be reacted with another (large) molecule comprising azido groups —N₃ under formation of a 1,2,3 triazole ring.

Compounds of formula (I) or (II) wherein R¹ is C(═O)R² and R² is (C), i.e. (CH₂)ₘ—N₃, can be reacted with another (large) molecule comprising ethynyl groups —C≡CH under formation of a 1,2,3 triazole ring.

If the compounds of formula (I) or (II) are not linked to other molecules, R¹ in NHR¹ is preferably H or C(═O)R², wherein R² is C₁₋C₆-alkyl, C₇₋C₂₂-arylalkyl O—C₁₋C₆-alkyl or O—C₇₋C₂₂-arylalkyl. In a preferred embodiment R¹ is H or C(═O)R², wherein R² is C₁₋C₆-alkyl or O—C₁₋C₆-alkyl, more preferably H or C(═O)R², wherein R² is C₁₋C₄-alkyl or O—C₁₋C₄-alkyl, like for instance C(═O)CH₃, C(═O)C(CH₃)₃, C(═O)(O)CH₃ or C(═O)(O)C(CH₃)₃.

In another embodiment a compounds of formula (I) or (II) is linked to a lipophilic compound to result in an amphiphilic compound of formula (I) or (II). Suitable lipophilic compounds are known in the art and contain a functional group that reacts with the NHR¹-group, preferably the NH₂-group, present in compounds of formula (I) and (II) and a lipophilic residue selected from the group of higher alkyl or higher alkenyl, preferably C₈-C₂₀-alkyl or C₈-C₂₀-alkenyl, arylalkyl or alkylaryl, cholesterol derivatives or bile salts. Suitable lipophilic compounds are for instance fatty acid chlorides like oleoyl chloride or stearyl chloride.

The resulting amphiphilic compound of formula (I) can then be reacted with for instance a salt containing a paramagnetic metal ion like for instance Gd(III)Cl₃ to result in a an amphiphilic compound of formula (II), hereinafter denoted “amphiphilic chelate”. The amphiphilic chelate can then be dispersed, optionally in combination with lipids or surfactants or a carrier oil phase to obtain a preferably monodisperse formulation of a chosen size, preferably a micellar size. Techniques for obtaining such dispersions are known in the art. Alternatively, the resulting amphiphilic compound of formula (I) is dispersed, optionally in combination with lipids or surfactants or a carrier oil phase to obtain a preferably monodisperse formulation of a chosen size, preferably a micellar size and the formulation is then reacted with for instance a salt containing a paramagnetic metal ion like for instance Gd(III)Cl₃ to result in a dispersed amphiphilic chelate, i.e. dispersed amphiphilic compound of formula (II).

As described earlier, capillary walls in tumours show permeability abnormalities, e.g. “leakiness” which is a result of tumour angiogenesis. By tailoring the size of the dispersed amphiphilic chelates in such a way that the dispersed amphiphilic chelate can pass through these leaky capillary walls into the tumour tissue (e.g. micellar size) it should be possible to obtain an MR contrast agent for tumour imaging.

In another embodiment, other contrast/imaging agents may be incorporated into such dispersed amphiphilic chelates, such as X-ray agents or air so that a combined MRI-X-ray or MRI-ultrasound agent would result.

Therefore, in a third aspect of the invention there is provided compounds of formula (I) or (II) which are linked via the NHR¹-group, preferably via the NH₂-group, to a lipophilic compound.

In yet another embodiment, compounds of formula (I) or (II) are linked via the NHR¹-group, preferably via the NH₂-group, to a nanoparticle surface. Preferred nanoparticles are metal oxide nanoparticles, gold nanoparticles, silver nanoparticles, silica nanoparticles, zinc nanoparticles or titanium nanoparticles. The choice of functional group, i.e. the NHR¹-group depends on the type of nanoparticle the compound of formula (I) and (II) is linked to. In a preferred embodiment, the nanoparticle is a gold nanoparticle and the NHR¹-group, preferably the NH₂-group, present in compounds of formula (I) and (II) is derivatised in such a way that it contains a thiol moiety and said thiol moiety can be used to link said compounds of formula (I) and (II) to the surface of a gold nanoparticle. In another embodiment, the NHR¹-group, preferably the NH₂-group, present in compounds of formula (I) and (II) is derivatised in such a way that it contains a trialkyloxysilane moiety and trialkyloxysilane can be used to link said compounds of formula (I) and (II) to the surface of a metal oxide nanoparticle. By linking compounds of formula (II) to a nanoparticle, multiple molecules of compounds of formula (II) are held rigidly relative to one another and this, together with the number of molecules of compounds of formula (II) per nanoparticle would ensure high relaxivity. In another embodiment, the nanoparticle itself has a function other than just being a carrier. In particular, the nanoparticle may have fluorescent properties thus resulting in a compound which is a combined MR—optical imaging agent.

Therefore, in a forth aspect of the invention there is provided compounds of formula (I) and (II) as defined above linked via the NHR¹-group, preferably via the NH₂-group to a nanoparticle surface. In a preferred embodiment, the nanoparticle is a metal oxide nanoparticle, a gold nanoparticle, a silver nanoparticle, a silica nanoparticle, a zinc nanoparticle or a titanium nanoparticle.

The compounds of formula (I) may be prepared by using 1,3,5,7-tetrakis(aminomethyl)adamantane as a starting material. 1,3,5,7-tetrakis(aminomethyl)adamantane may be obtained as described by G. S. Lee et al., Org. Lett. Vol. 6, No. 11, 2004, 1705-1707. Briefly, adamantane is reacted with AlBr₃/Br₂ to tetrabromoadamantane whose subsequent photolysis with NaCN in DMSO results in tetracyanoadamantane. 1,3,5,7-tetrakis(aminomethyl)adamantane is then obtained by reduction of tetracyanoadamantane with monochloroborane and reaction with dry methanolic HCl.

In a subsequent step, a mono-protected 1,3,5,7-tetrakis(aminomethyl)adamantane is produced which is then coupled to a compound of formula (IV) comprising X which is protected and a leaving group:

X^{Z}—C(O)—Y (IV)

wherein
X^{Z} is X as defined before wherein the hydroxyl groups which are bound to X are protected; and
Y is a leaving group, preferably a halide, a mixed anhydride, an activated ester such as O-succinimide or an activated amide such as imidazolide.

The hydroxyl group(s) present in X, i.e. attached to the ring system need to be protected. Suitable protecting groups for hydroxyl groups are well known in the art and are for instance described in “Protecting Groups in Organic Synthesis”, Theodora W. Greene and Peter G. M. Wuts, published by John Wiley & Sons Inc. Examples of suitable groups of protecting groups for hydroxyl groups include tert.-butyl groups or benzyl, with benzyl being preferred.

If X contains one or more substituents R, hydroxyl groups present in R may or may not be protected. If R comprise other reactive groups than the aforementioned hydroxyl groups, e.g. such as amine groups, such groups need to be protected as well. Again suitable protecting groups are well known in the art.

The reaction of mono-protected 1,3,5,7-tetrakis(aminomethyl)adamantane with compounds of formula (IV) is preferably conducted in organic solvent(s) such as dichloromethane or tetrahydrofuran (THF) under anhydrous conditions but for some reagents, an aqueous solution may be used.

The reaction is illustrated in reaction scheme 1:

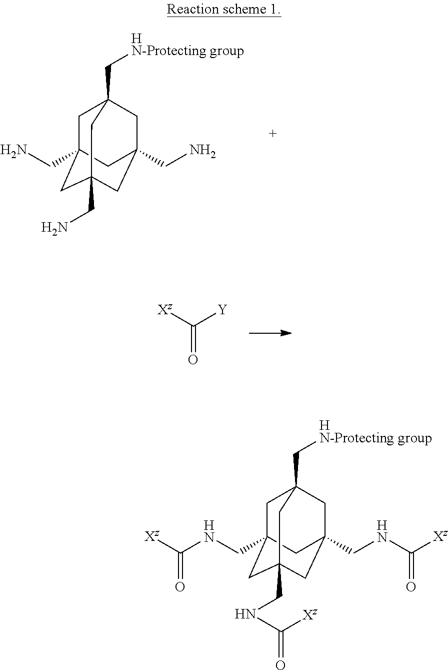

Suitable protecting groups for amines are known in the art and a mono-protected 1,3,5,7-tetrakis(aminomethyl)adamantane can be obtained by reacting 1 equivalent 1,3,5,7-tetrakis(aminomethyl)adamantane with ¼ equivalent of a precursor, e.g. an acyl chloride or anhydride, of the chosen protection group. A preferred precursor of such a protecting group is benzyl chloroformate or BOC anhydride (di-tert-butyl dicarbonate)

Compounds of formula (IV) are also known and may be prepared by known methods. For example, compounds of formula (IV) in which X is a group of formula (IIIa) are designated compounds of formula (IVa):

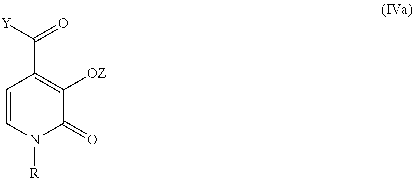

wherein R and Y are as defined above and Z is a protecting group for OH as described above.

Compounds (IVa) may be prepared by reacting compounds of formula (V) which are well known in the art:

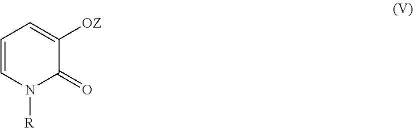

wherein R and Z are as defined above, with carbon dioxide in the presence of a base. A suitable method for this reaction is set out in U.S. Pat. No. 5,624,901.

Other compounds of formula (IV) which have a different X group, for example an X group of formula (IIIb), (IIIe), (IIIf) and (IIIg) can be prepared by methods similar to those above or methods known to those skilled in the art and set out in, for example US-A-2003/0095922, Z. Liu et al., Bioorg. Med. Chem. 9 (2001), 563-573, S. Piyamongkol et al., Tetrahedron Letters 46 (2005), 1333-1336, V. Pierre et al., J. Am. Chem. Soc. 2006, 128, 5344-5345, J. Xu et al., J. Am. Chem. Soc. 1995, 117, 7245-7246, D. Doble et al., J. Am. Chem. Soc. 2001, 123, 10758-10759, M. Allen et al., J. Am. Chem. Soc. 2006, 128, 6534-6535, M. Seitz et al., Inorg. Chem. 2007, 46, 351-353, K. Clarke Jurchen et al., Inorg. Chem. 2006, 45, 1078-1090, B. O'Sullivan et al., Inorg. Chem. 2003, 42, 2577-2583, D. Doble et al., Inorg. Chem. 2003, 42, 4930-4937, S. Dhungana et al., Inorg. Chem. 2001, 40, 7079-7086, A. Johnson et al., Inorg. Chem. 2000, 39, 2652-2660, S. Cohen et al., Inorg. Chem. 2000, 39, 4339-4346.

Compounds of formula (IVc) which have an X group of formula (IIIc):

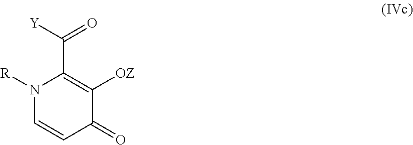

are preferably produced as illustrated in reaction scheme 2, wherein Y′ denotes a precursor of Y:

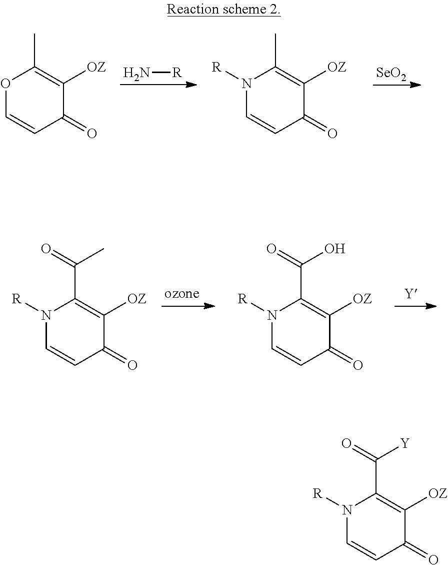

In a subsequent reaction to the reaction illustrated in reaction scheme 1, the protecting groups Z and the amino protecting groups are removed by methods known in the art and compounds of formula (I) are obtained. Said subsequent reaction is illustrated in reaction scheme 3:

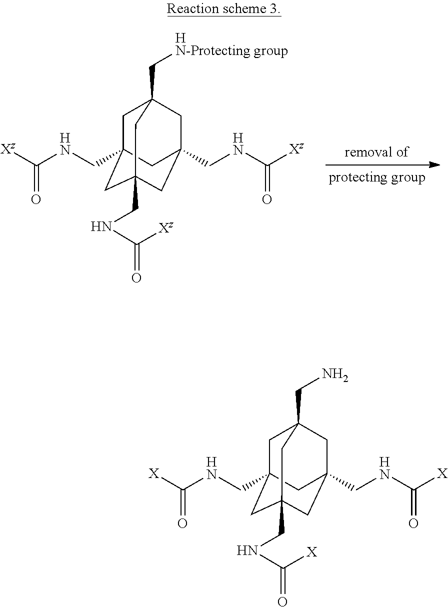

In a first embodiment, the removal of said protecting groups is done in a two-step procedure. In a first step, the amino protecting group is removed and the free amino group may be reacted with a suitable compound to give compounds of formula (I) wherein R¹ is different from H, e.g. to link compounds of formula (I) to other molecules, for instance larger molecules like proteins, polymers or dendrimers. In a second step, the protecting groups Z are removed. Said first embodiment is preferred if compounds of formula (I) are linked to other molecules. In a second embodiment, the removal of said protecting groups is done in a one step procedure, i.e. the amino protecting group and the protecting groups Z are removed simultaneously. Said second embodiment is preferred if the compound of formula (I) is not linked to another molecule.

Thus, in another aspect the invention provides a method for producing the compound of formula (I) wherein R¹ is H by reacting a mono-protected 1,3,5,7-tetrakis(aminomethyl)adamantane with a compound of formula (IV)

X^{Z}—S(O)—Y (IV)

wherein
X^{Z} is X as defined earlier and wherein the hydroxyl groups which are bound to X are protected; and
Y is a leaving group; and
   removing the amino protecting group of said mono-protected 1,3,5,7-tetrakis(aminomethyl)adamantane and optionally the hydroxyl protecting groups of X^{Z}.

Compounds of formula (I) wherein NHR¹ is NH₂ are useful starting materials for the production of compounds of formula (II) wherein R¹ is different from H and which are for instance to be linked to other molecules, e.g. larger molecules like proteins, polymers or dendrimers. If compounds of formula (I) wherein NHR¹ is NH₂ are to be used as such starting materials, the hydroxyl protecting groups of X^{Z} are preferably not removed.

Thus, in yet another aspect the invention provides a method for producing a compound of formula (I) wherein R¹ is C(═O)R² and R² is C₁-C₆-alkyl, C₆-C₁₅-aryl, C₇-C₂₂-arylalkyl, O—C₁-C₆-alkyl, O—C₇-C₂₂-arylalkyl, (CH₂)ₘ—C≡CH or (CH₂)ₘ—N₃ by
a) reacting a mono-protected 1,3,5,7-tetrakis(amino-methyl)adamantane with a compound of formula (IV)

X^{Z}—S(O)—Y (IV)
wherein
X^{Z} is X as defined earlier and wherein the hydroxyl groups which are bound to X are protected; and
Y is a leaving group;
b) removing the amino protecting group of said mono-protected 1,3,5,7-tetrakis(aminomethyl)adamantane;
c) reacting the product obtained with a compound of formula (VI); and

Y—C(═O)R² (VI)
d) removing the hydroxyl protecting groups of X^{Z}.

In yet another aspect the invention provides a method for producing a compound of formula (I) wherein R¹ is C(═O)R² and R² is (CH₂)ₙ—(C₆H₄)—NCS by
a) reacting a mono-protected 1,3,5,7-tetrakis(amino-methyl)adamantane with a compound of formula (IV)

X^{Z}—C(O)—Y (IV)
wherein
X^{Z} is X as defined earlier and wherein the hydroxyl groups which are bound to X are protected; and
Y is a leaving group;
b) removing the amino protecting group of said mono-protected 1,3,5,7-tetrakis(aminomethyl)adamantane;
c) reacting the product obtained with a compound of formula (VI_{A}*)

Y—C(—O)—(CH₂)ₙ—(C₆H₄)—NO₂ (VI_{A}*)
wherein n and Y are as defined above;
d) reducing the nitro group to an amino group;
e) reacting the amino group with thiophosgene to give the isothiocyanate group —NCS; and
f) removing the hydroxyl protecting groups of X^{Z}.

To produce compounds of formula (II) from compounds of formula (I) the compounds of formula (I) obtained in the reaction illustrated above are reacted with a chosen paramagnetic metal ion M, preferably in the form of its salt, e.g. nitrate, chloride, acetate and sulphate salts, in water as a solvent. Alternatively, an oxide of said chosen paramagnetic metal ion M may be used, e.g. Gd₂O₃, and a solution of the compound of formula (I) is then stirred with said oxide. This method is often preferred since it avoids the problem of free residual paramagnetic metal ions being present in the reaction product.

Thus in a sixth aspect the invention provides a method for producing a compound of formula (II) by reacting a compound of formula (I) with a paramagnetic metal ion, preferably in the form of its salt or in the form of its oxide.

Compounds of formula (I) linked to other molecules via the NHR¹-group can be prepared by methods known in the art. If for instance said other molecule is a peptide, polypeptide or protein, compounds of formula (I) can be readily linked to suitable functional groups in said other molecules, e.g. carboxyl groups. It may be necessary to activate the functional groups in said other molecules, e.g. generating an acyl chloride from a carboxyl group. Methods to activate functional groups in order to enhance their reactivity are known to the skilled person in the art (see for example Sandler and Karo, eds. Organic Functional Group preparation, Academic Press, San Diego 1998).

Compounds of formula (II) linked to other molecules via the NHR¹-group can be prepared by methods known in the art. In one embodiment, a compound of formula (I) is linked to another molecule via the NHR¹-group as described in the previous paragraph and the reaction product obtained is reacted with a chosen paramagnetic metal ion M to result in a compound of formula (II) linked to said another molecule. In another embodiment a compound of formula (II) is directly linked to another molecule via the NHR¹-group as described in the previous paragraph.

As previously discussed, if compounds of formula (I) or (II) are linked to large molecules like proteins, polymers or dendrimers, click chemistry is preferred to achieve said linking. Thus, in a preferred embodiment, R¹ is C(═O)R² and R² is as follows and * denotes the attachment point of R² to C of group C(═O)R²:

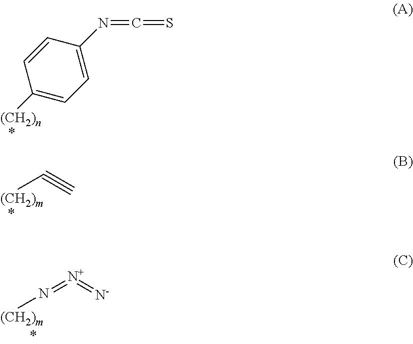

Compounds of formula (I) wherein R¹ is C(═O)R² and R² is (B), i.e. (CH₂)ₘ—C≡CH or (C), i.e. (CH₂)ₘ—N₃ can be prepared by reacting a compound of formula (VI)

Y—C(═O)R² (VI)

wherein R² is (B) or (C) as defined above and Y is a leaving group as defined above with an optionally protected compound of formula (I) wherein NHR¹ is NH₂.

Compounds of formula (VI) wherein R² is (B) may be prepared by for instance reacting an ω-alkynoic acid HOOC—(CH₂)ₘ—C≡CH with N-hydroxy-succinimide in the presence of a coupling agent such as DCC (N,N′-dicyclohexylcarbodiimide).

Compounds of formula (VI) wherein R² is (C) may be prepared by for instance reacting an ω-azido carboxylic acid HOOC—(CH₂)ₘ—N₃ with N-hydroxy-succinimide in the presence of a coupling agent such as DCC (N,N′-dicyclohexylcarbodiimide).

Compounds of formula (I) wherein R¹ is C(═O)R² and R² is (A), i.e. (CH₂)ₙ—(C₆H₄)—NCS can be prepared by
a) reacting a compound of formula (VI_{A}*)

Y—C(═O)—(CH₂)ₙ—(C₆H₄)—NO₂ (VI_{A}*)
wherein
n is defined as above and
Y is a leaving group as defined above
with an optionally protected compound of formula (I) wherein NHR¹ is NH₂,
b) reducing the nitro group to an amino group; and
c) reacting the amino group with thiophosgene to give the isothiocyanate group —N═C═S.

Compounds of formula (VI_{A}*) may be prepared by for instance reacting a carboxylic acid of the following formula HOOC—(CH₂)ₙ—(C₄H₆)—NO₂ with N-hydroxysuccinimide in the presence of a coupling agent such as DCC (N,N′-dicyclohexylcarbodiimide).

Compounds of formula (VI) and (VI_{A}*) can be reacted with compounds of formula (I) as shown in reaction scheme 4:

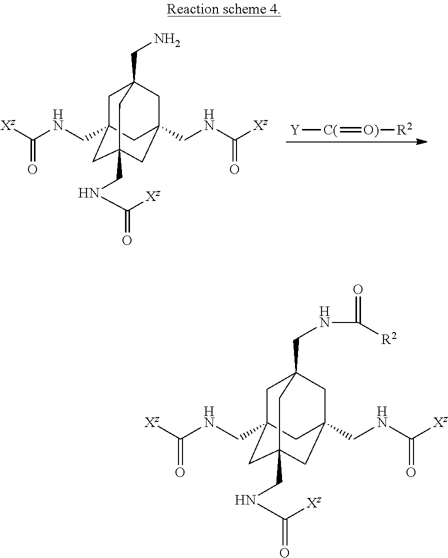

In a subsequent step, compounds of formula (I) wherein NHR¹ is C(═O)R² and R² is (A), (B) or (C) can be linked to another molecule, preferably a large molecule like a protein, polymer or dendrimer.

Compounds of formula (I) wherein R¹ is C(═O)R² and R² is (A) are readily linked to other (large) molecules comprising amino groups. This reaction is shown in reaction scheme 5A:

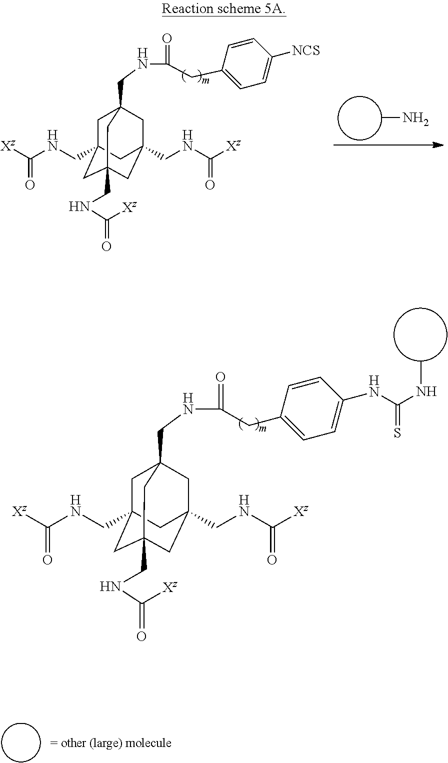

Compounds of formula (I) wherein R¹ is C(═O)R² and R² is (B) are readily linked to other (large) molecules comprising azido groups. This reaction is shown in reaction scheme 5B:

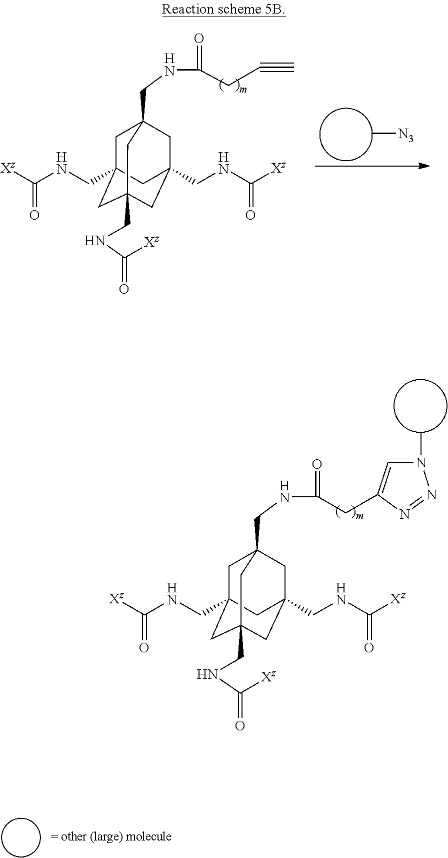

Compounds of formula (I) wherein R¹ is C(═O)R² and R² is (C) are readily linked to other (large) molecules comprising ethynyl groups. This reaction is shown in reaction scheme 5C:

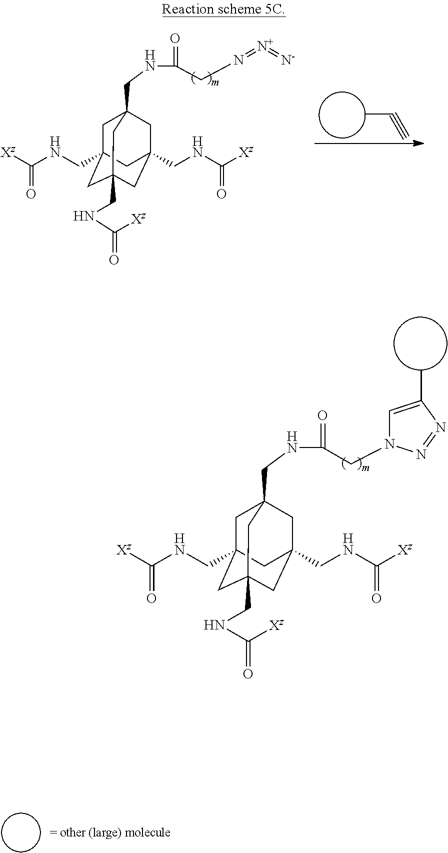

Compounds of formula (II) and compounds of formula (II) linked to other molecules, preferably to natural or synthetic peptides, peptidomimetics, polypeptides, proteins, antibodies, natural or synthetic polymers, dendrimers, lipophilic compounds or nanoparticles may be used as MR contrast agents.

For this purpose, the compounds of formula (II) and compounds of formula (II) linked to other molecules are formulated with conventional physiologically tolerable carriers like aqueous carriers, e.g. water and buffer solutions, and optionally with excipients. The resulting composition is denoted “MR contrast medium”.

Thus in a further aspect the invention provides a composition comprising a compound of formula (II) or a compound of formula (II) linked to other molecules and at least one physiologically tolerable carrier. Said composition may be used as MR contrast medium in MRI.

To be used as MR contrast medium in MRI of the human and non-human animal body, said MR contrast medium needs to be suitable for administration to said body. Suitably, the compounds of formula (II) or compounds of formula (II) linked to other molecules and optionally pharmaceutically acceptable excipients and additives may be suspended or dissolved in at least one physiologically tolerable carrier e.g. water or buffer solution(s). Suitable additives include for example physiologically compatible buffers like tromethamine hydrochloride, chelators such as DTPA, DTPA-BMA or compounds of formula (I), weak complexes of physiologically tolerable ions such as calcium chelates, e.g. calcium DTPA, CaNaDTPA-BMA, compounds of formula (I) wherein X forms a complex with Ca²⁺ or Ca/Na salts of compounds of formula (I), calcium or sodium salts like calcium chloride, calcium ascorbate, calcium gluconate or calcium lactate. Excipients and additives are further described in e.g. WO-A-90/03804, EP-A-463644, EP-A-258616 and U.S. Pat. No. 5,876,695, the content of which are incorporated herein by reference.

Another aspect of the invention is the use of a composition comprising a compound of formula (II) or a compound of formula (II) linked to another molecule and at least one physiologically tolerable carrier as MR imaging medium.

Yet another aspect of the invention is a method of MR imaging wherein a composition comprising a compound of formula (II) or a compound of formula (II) linked to another molecule and at least one physiologically tolerable carrier is administered to a subject and the subject is subjected to an MR examination wherein MR signals are detected from the subject or parts of the subject into which the composition distributes and optionally MR images and/or MR spectra are generated from the detected signals. In a preferred embodiment, the subject is a living human or non-human animal body.

In a further preferred embodiment, the composition is administered in an amount which is contrast-enhancing effective, i.e. an amount which is suitable to enhance the contrast in the method of MR imaging.

In another preferred embodiment, the subject is a living human or non-human animal being and the method of MR imaging is a method of MR tumour detection or a method of tumour delineation imaging.

In another aspect, the invention provides a method of MR imaging wherein a subject which had been previously administered with a composition comprising a compound of formula (II) or a compound of formula (II) linked to another molecule and at least one physiologically tolerable carrier is subjected to an MR examination wherein MR signals are detected from the subject or parts of the subject into which the composition distributes and optionally MR images and/or MR spectra are generated from the detected signals.

The term “previously been administered” means that any step requiring a medically-qualified person to administer the composition to the patient has already been carried out before the method of MR imaging and/or MR spectroscopy according to the invention is commenced.

The invention will now be described in greater detail by way of the following non-limiting examples.

### EXAMPLES

### Example 1

### Synthesis of 1,3,5,7-tetrakis(aminomethyl)adamantane (3)

### 1a Tetrabromoadamantane (1)

Adamantane was brominated with bromine/AlBr₃ as described in G. S. Lee et al., Org. Lett. Vol. 6, No. 11, 2004, 1705-1707, scheme 2^{a}, (a). The title compound (1) was obtained in 85% yield.

### 1b Tetracyanoadamantane (2)

Compound (1) of Example 1a was reacted with sodium cyanide in DMSO under irradiation at 254 nm as described in G. S. Lee et al., Org. Lett. Vol. 6, No. 11, 2004, 1705-1707, scheme 2^{a}, (c). Tetracyanoadamantane was obtained in 63% yield.

### 1c 1,3,5,7-tetrakis(aminomethyl)adamantane (3)

Compound (2) of Example 1b was reduced with monochloroborane-dimethylsulphide in THF as described by G. S. Lee et al., Org. Lett. Vol. 6, No. 11, 2004, 1705-1707, scheme 2^{a}, (f). The title compound (3) was obtained in 98% yield.

### Example 2

### Protection of 1,3,5,7-tetrakis(aminomethyl)adamantane (4)

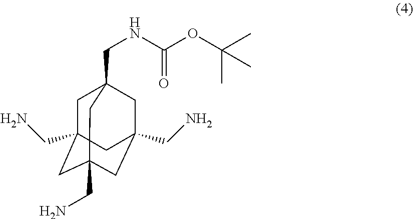

Compound (3) was dissolved in dichloromethane and reacted with 1 equivalent BOC anhydride. The reaction was allowed to stand at room temperature over night. The reaction was concentrated in vacuo and chromatographed on a reverse phase column in 0-30% acetonitrile in water. The fraction containing the protected 1,3,5,7-tetrakis(aminomethyl)adamantane (4) was collected.

### Example 3

### Coupling of the N-hydroxysuccinimide ester of 2-carboxy-3-benzyloxy-pyran-4-(1H)-one (5) to (4) to result in (6)

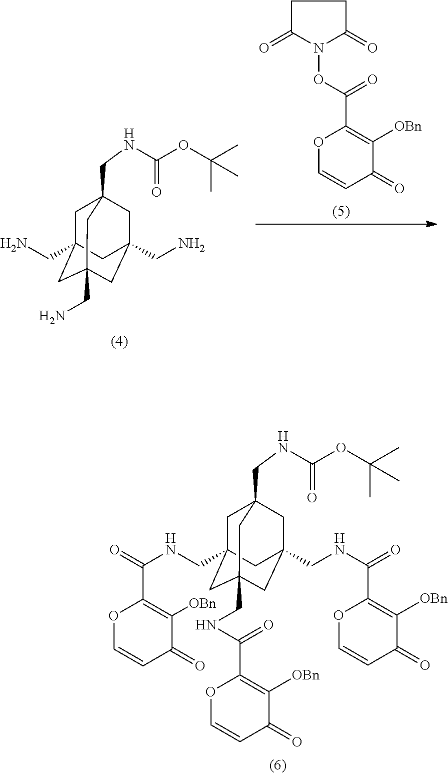

3.5 equivalents of 2-carboxy-3-benzyloxy-pyran-4-(1H)-one N-hydroxy succinimide ester (5) which was prepared as described in by D. Puerta et al., J. Am. Chem. Soc. Vol. 128, No. 7, 2006, 2222-2223, were combined with 1 equivalent of (4) and 4 equivalents of triethylamine in THF. The reaction mixture was stirred over night at room temperature. The solvent was removed in vacuo. The residue was dissolved in dichloromethane and washed with 10% aqueous potassium carbonate. The dichloromethane was evaporated and the crude reaction product (6) was purified by chromatography on silica in a gradient of 5-10% methanol in dichloromethane.

### Example 4

### Reaction of (6) with 1-amino-2,3,4-butanetriol to result in (7)

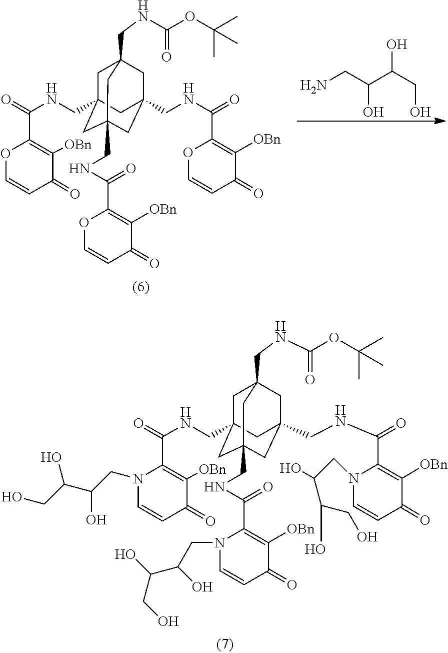

To a solution of 1 equivalent (6) in methanol was added 8 equivalents 1-amino-2,3,4-butanetriol prepared as described in EP-A1-0675105 on page 10, example Eiii) and the reaction mixture was heated under reflux for 3 hours. The solvent was removed under reduced pressure and the residue was purified by column chromatography on silica using dichloromethane/methanol 9:1 to give reaction product (7).

### Example 5

### Reaction of (6) with 3-amino-1,2-propanediol to result in (8)

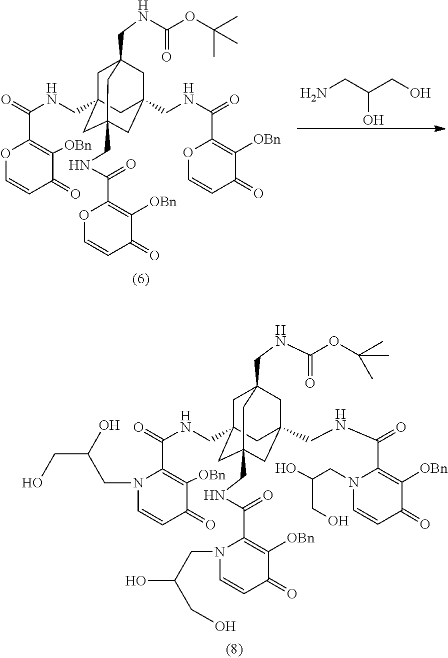

To a solution of 1 equivalent (6) in methanol was added 8 equivalents 3-amino-1,2-propanediol and the reaction mixture was heated under reflux for 3 hours. The solvent was removed under reduced pressure and the residue was purified by column chromatography on silica using dichloromethane/methanol 9:1 to give reaction product (8).

### Example 6

### Removal of the BOC Protecting Groups of (6) to Result in (9)

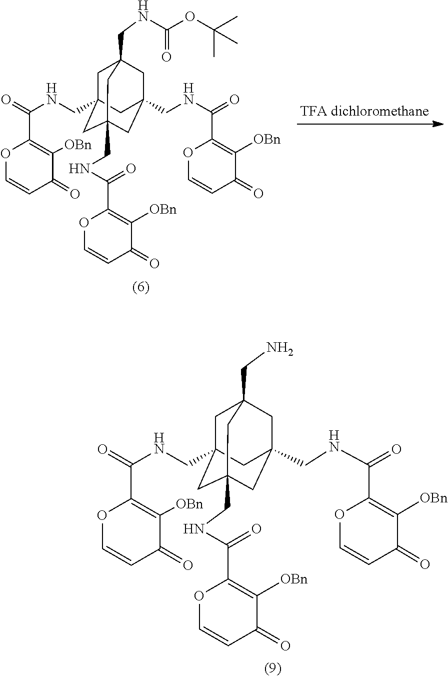

To a solution of (6) in dichloromethane was added trifluoroacetic acid. The reaction was followed by NMR. When complete the reaction was concentrated in vacuo to give reaction product (9) as the TFA salt.

### Example 7

### Removal of the BOC Protecting Groups of (7) to Result in (10)

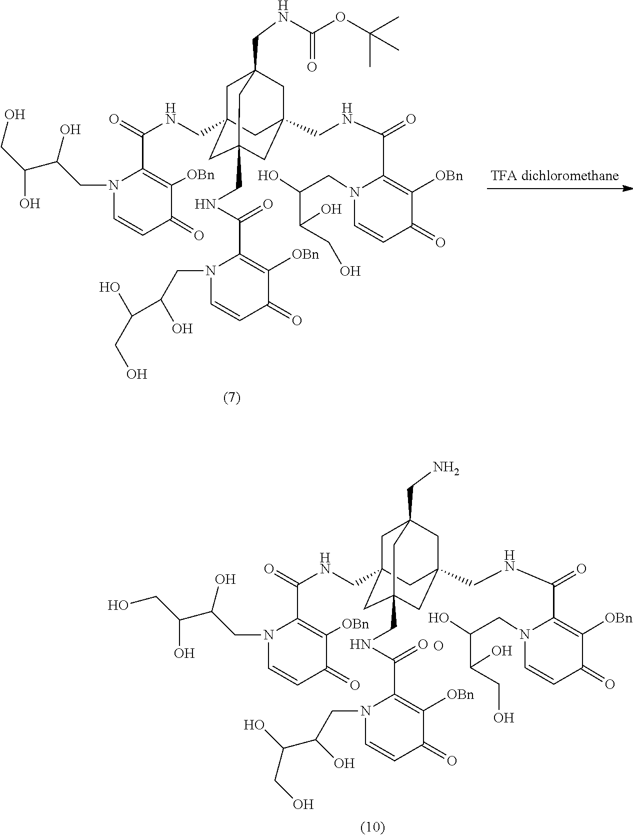

To a solution of (7) in dichloromethane was added TFA. The reaction was followed by NMR. When complete the solution was concentrated in vacuo to give reaction product (10) as the TFA salt.

### Example 8

### Removal of the BOC Protecting Groups of (8) to Result in (11)

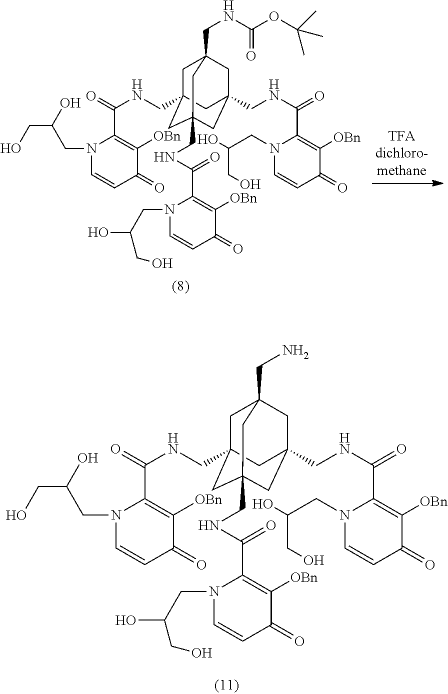

To a solution of (8) in dichloromethane was added trifluoroacetic acid. The reaction was followed by NMR. When complete the solution was concentrated in vacuo to give reaction product (11) as the TFA salt.

### Example 9

### Synthesis of the N-hydroxysuccinimide ester of 1-(2,3-benzyloxypropyl)-2-carboxy-3-benzyloxypyridine-4-one (16)

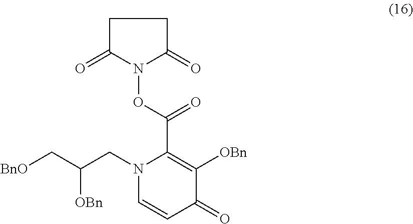

### 9a 1-(2,3-dihydroxypropyl)-2-methyl-3-benzyloxypyridine-4-one (12)

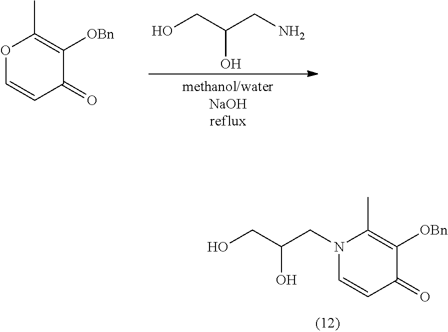

2-methyl-3-benzyloxypyran-4(1H)-one which was obtained by benzyl protection as described by P. Pace et al., Bioorg. Med. Chem. Lett. 2004, 14, 3257-3261 was refluxed with 2 equivalents of 3-amino-1,2-propanediol in a solution of methanol/water (1:1) with 0.16 equivalents of sodium hydroxide to give the title compound (12)

### 9b 1-(2,3-benzyloxypropyl)-2-methyl-3-benzyloxypyridine-4-one (13)

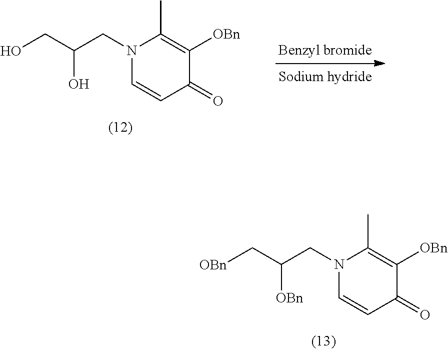

Compound (12) in dry THF was refluxed with 2.2 equivalents of benzyl bromide and 2.5 equivalents of sodium hydride. The reaction mixture was concentrated and re-dissolved in dichloromethane, washed with water and after drying concentrated to give title compound (13).

### 9c 1-(2,3-benzyloxypropyl)-2-formyl-3-benzyloxypyridine-4-one (14)

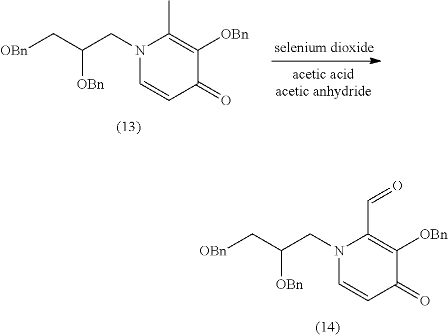

The title compound (14) was obtained by oxidation of compound (13) with 1.8 equivalents of selenium dioxide in acetic acid/acetic anhydride (1:1) at reflux temperature for 4 h. The solvent was then removed in vacuo. The residue was dissolved in ethyl acetate, washed with base to remove excess acetic acid, dried over sodium sulphate and concentrated. The concentrate was then chromatographed on silica in a gradient of 5% methanol in ethyl acetate.

### 9d 1-(2,3-benzyloxypropyl)-2-carboxy-3-benzyloxypyridine-4-one (15)

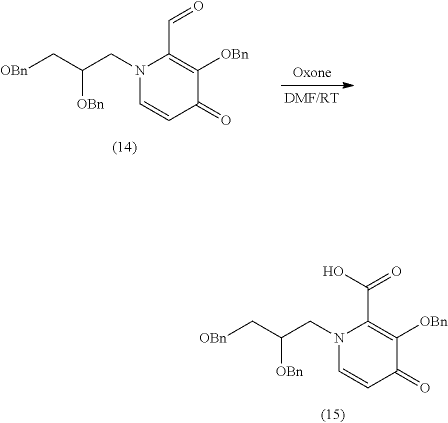

The title compound (15) was obtained by treating a solution of compound (14) in DMF with ozone at room temperature for 12 h. The product (15) was obtained by treating the reaction with water when it precipitated out.

### 9e NHS-ester of 1-(2,3-benzyloxypropyl)-2-carboxy-3-benzyloxypyridine-4-one (16)

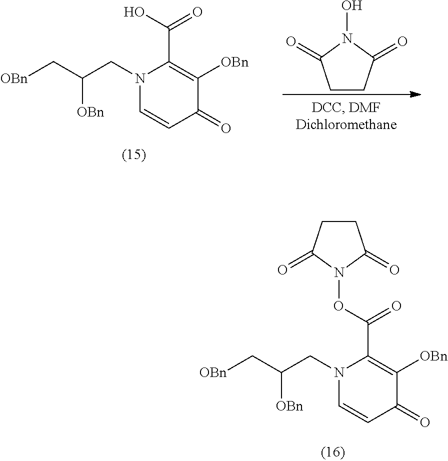

The title compound (16) was obtained by reaction of compound (15) with one equivalent of N-hydroxysuccinimide and one equivalent of N,N′-dicyclohexylcarbodiimide (DCC) in DMF and dichloromethane. The product was isolated from the reaction mixture by chromatography on silica in 5% methanol in ethyl acetate.

### Example 10

### Coupling of (16) to (4) to Give (17)

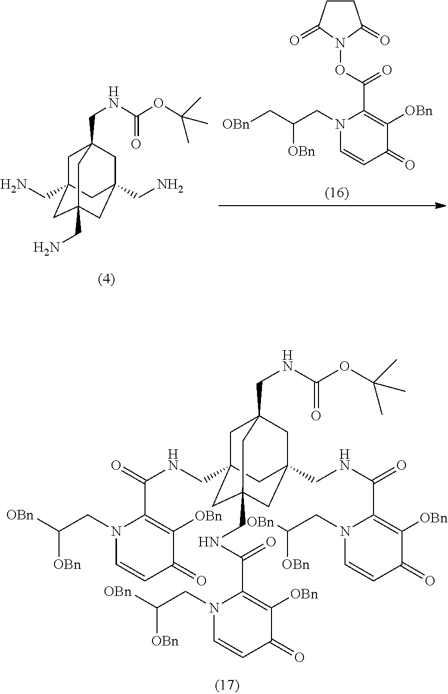

3.5 equivalents of (16) were combined with 1 equivalent of (4) and 4 equivalents of triethylamine in THF. The reaction mixture was stirred over night at room temperature. The solvent was removed in vacuo. The residue was dissolved in dichloromethane and washed with 10% aqueous potassium carbonate. The dichloromethane was evaporated and the crude reaction product (17) was purified by chromatography on silica in a gradient of 5-10% methanol in dichloromethane.

### Example 11

### Removal of the BOC Protecting Groups of (17) to Result in (18)

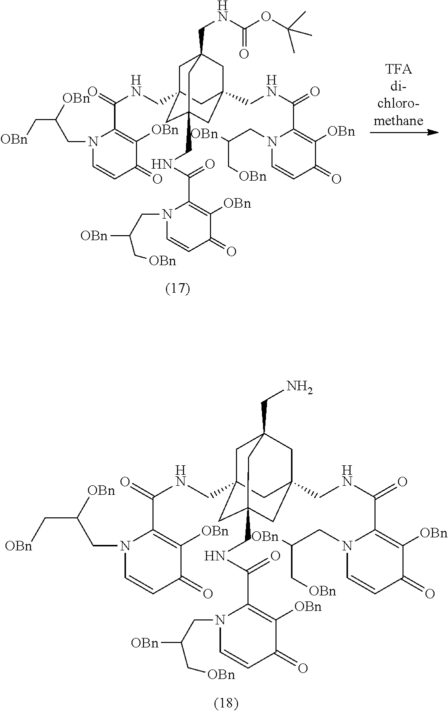

To a solution of (17) in dichloromethane was added trifluoroacetic acid. The reaction was followed by NMR. When complete the solution was concentrated in vacuo to give reaction product (18) as the TFA salt.

### Example 12

### Removal of the Benzyl Protecting Groups of (18) to Result in (19)

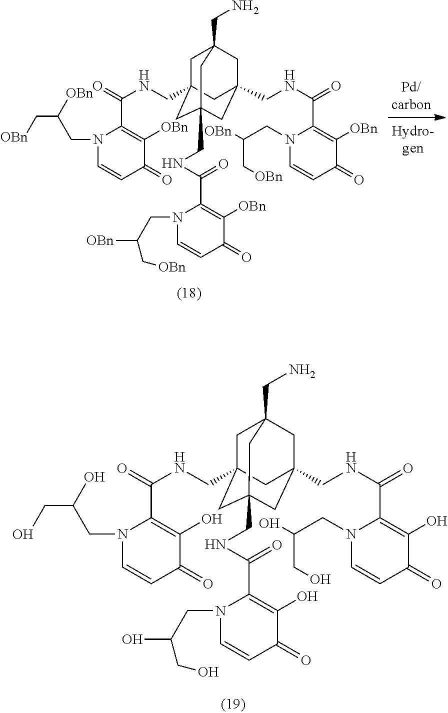

To a solution of (18) in methanol was added palladium on charcoal and the mixture was stirred under an atmosphere of hydrogen for several hours. The reaction mixture was then filtered and concentrated in vacuo to give (19)

### Example 13

### Formation of a Paramagnetic Chelate (20) by Complexing Gd³⁺ with the Chelator (19)

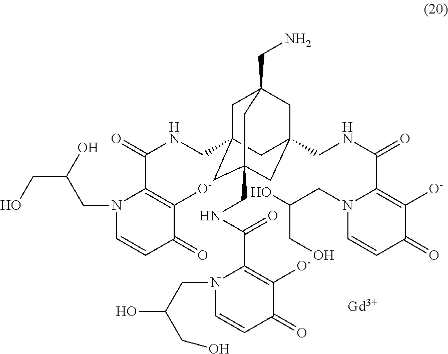

To a solution of compound (19) in water and methanol was added Gd(III)acetate. The reaction mixture was heated to reflux for 2 hours and then concentrated under reduced pressure. The residue was purified by reverse phase chromatography eluting with a gradient of methanol in water to give the paramagnetic chelate (20)

### Example 14

### Removal of the Benzyl Protecting Group of (9) to Result in (21)

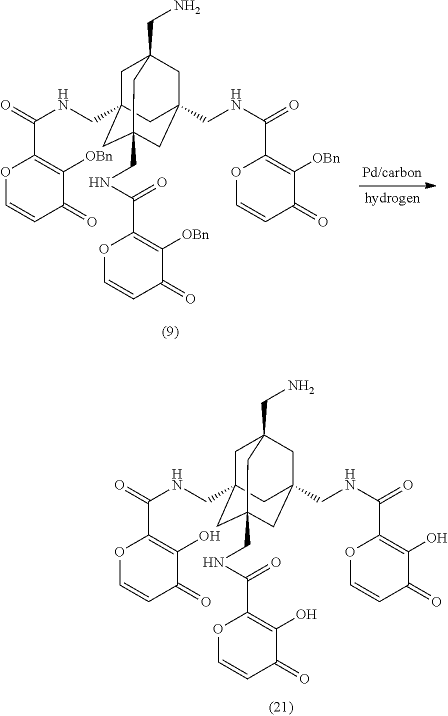

To a solution of (9) in methanol was added palladium on charcoal and the mixture was stirred under an atmosphere of hydrogen for several hours. The reaction mixture was then filtered and concentrated in vacuo to give (21)

### Example 15

### Formation of a Paramagnetic Chelate (22) by Complexing Gd³⁺ with the Chelator (21)

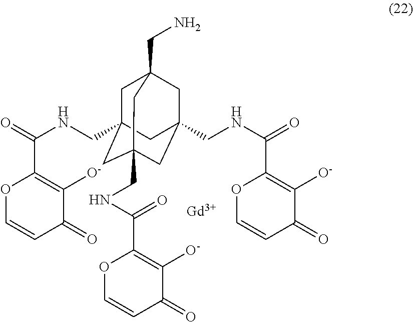

To a solution of compound (21) in water and methanol was added Gd(III)acetate. The reaction mixture was heated to reflux for 2 hours and then concentrated under reduced pressure. The residue was purified by reverse phase chromatography eluting with a gradient of methanol in water to give the paramagnetic chelate (22)

### Example 16

### Removal of the Benzyl Protecting Group of (10) to Result in (23)

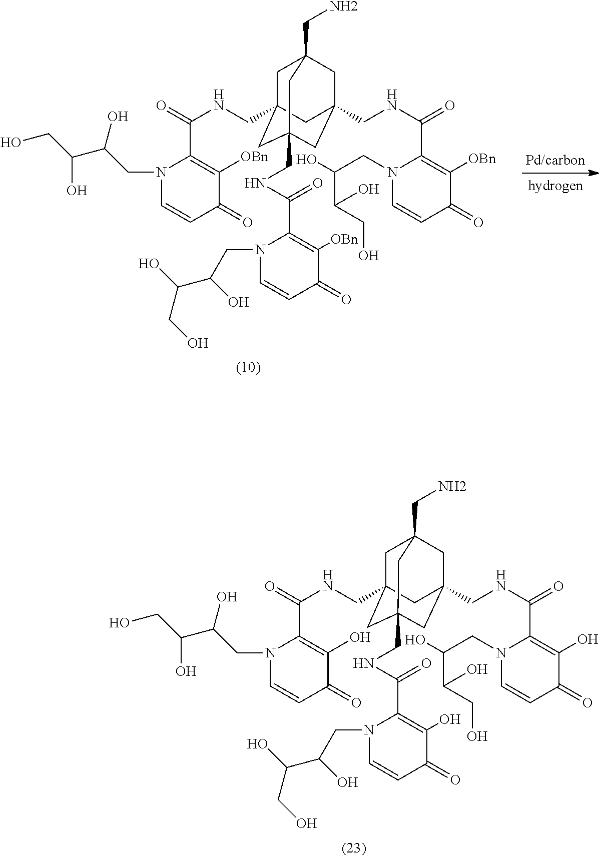

To a solution of (10) in methanol was added palladium on charcoal and the mixture was stirred under an atmosphere of hydrogen for several hours. The reaction mixture was then filtered and concentrated in vacuo to give (23)

### Example 17

### Formation of a Paramagnetic Chelate (24) by Complexing Gd³⁺ with the Chelator (23)

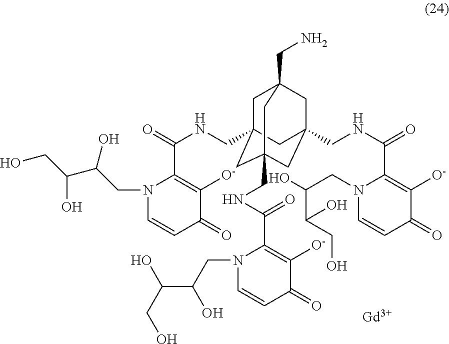

To a solution of compound (23) in water and methanol was added Gd(III)acetate. The reaction mixture was heated to reflux for 2 hours and then concentrated under reduced pressure. The residue was purified by reverse phase chromatography eluting with a gradient of methanol in water to give the paramagnetic chelate (24)

### Example 18

### Removal of the Benzyl Protecting Group of (11) to Result in (19)

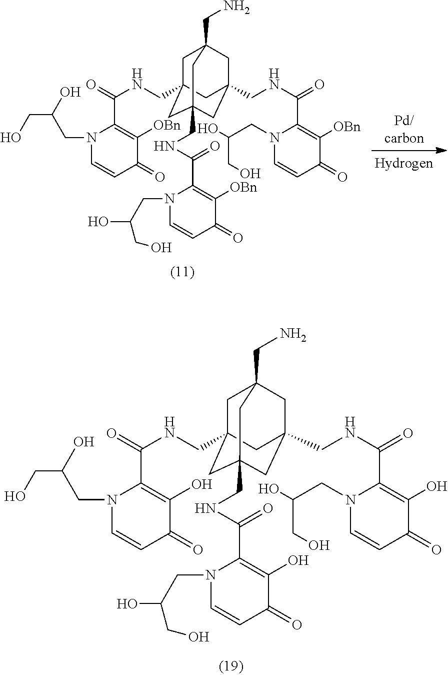

To a solution of (11) in methanol was added palladium on charcoal and the mixture was stirred under an atmosphere of hydrogen for several hours. The reaction mixture was then filtered and concentrated in vacuo to give (19) which was used to synthesize the paramagnetic chelate (20) as described in Example 13.

### Example 19

### Synthesis of the N-hydroxysuccinimide ester of 6-azidohexanoic acid (25)

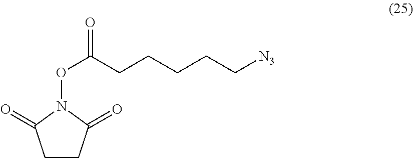

As described in C. Grandjean et al., J. Org. Chem. 2005, 70, 7123-7126, bromohexanoic acid was reacted at 85° C. with 2 equivalents of sodium azide in DMF to result in 6-azidohexanoic acid which after extraction in dichloromethane was reacted with 1 equivalent of N-hydroxysuccinimide in the presence of 1 equivalent of N-ethyl-N′-dimethylaminopropylcarbodiimide (EDC) to give after washing with 1 N hydrochloric acid and aqueous sodium hydrogen carbonate the title compound (25)

### Example 20

### Reaction of (9) with the NHS-Ester (25) to Result in (26)

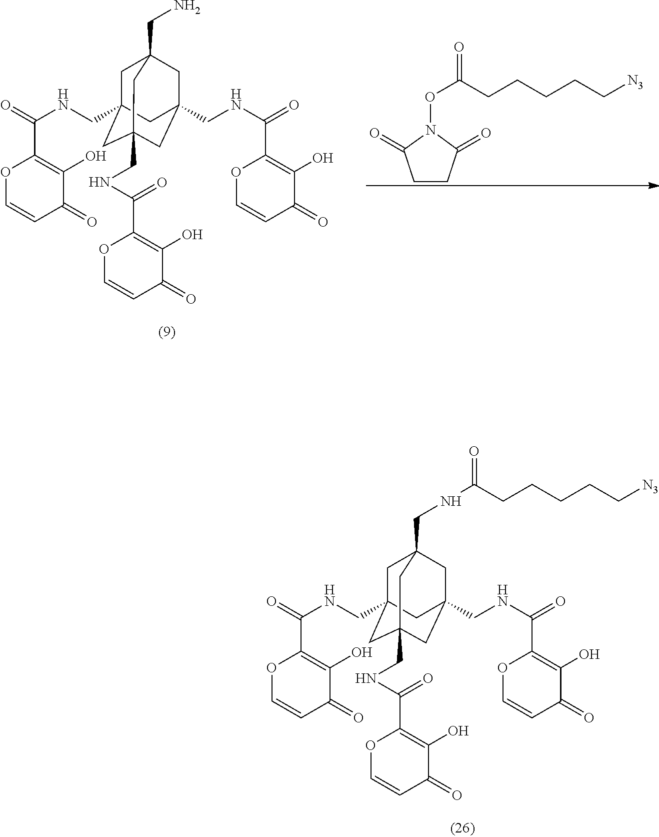

To a solution of 1 equivalent (9) in THF was added 1.3 equivalents (25) and 1.5 equivalents triethylamine. The reaction was stirred at room temperature for 12 h. 1 equivalent of water was added to hydrolyse the excess of NHS-ester and the reaction was stirred at room temperature for another hour. The reaction was then concentrated in vacuo, the residue was dissolved in dichloromethane and washed with aqueous potassium carbonate solution to obtain title compound (26).

Compounds (27) and (28) were synthesized as described above using compounds (10) and (11) as starting materials.

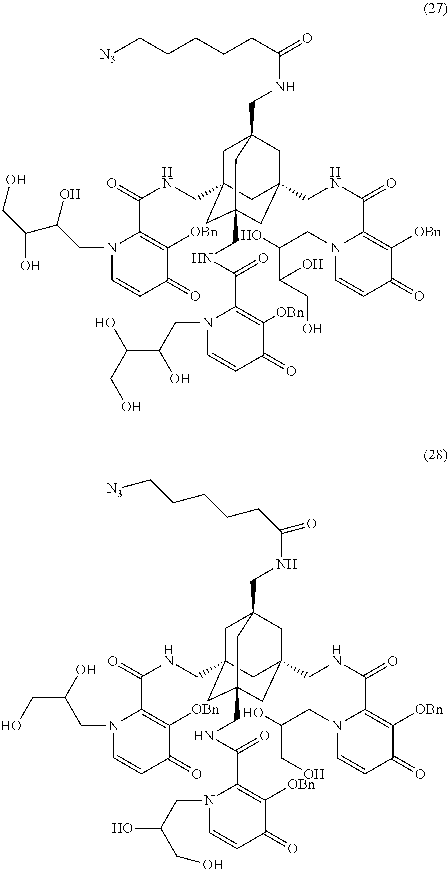

### Example 21

### Reaction of (26) with a Dendrimer Containing Acetylene Groups to Result in (29), a Compound of Formula (I) Linked to a Dendrimer

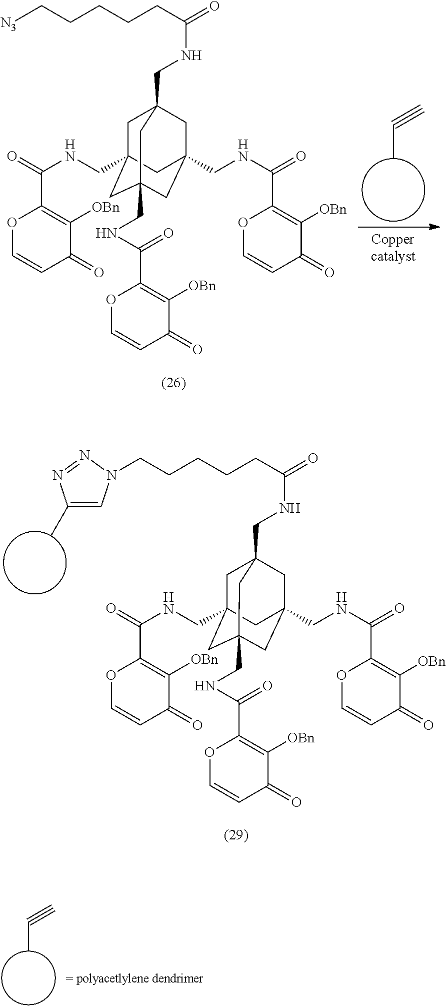

An excess of compound (26) was dissolved in THF and a dendrimer containing acetylene groups was added. The azide-acetylene cycloaddition was carried out in the presence of ascorbic acid using CuSO₄ as a catalyst as reviewed by P. Wu et al, Aldrichimica Acta 40(1), 2007, 7-15. The reaction mixture was allowed to stir over night and then concentrated in vacuo. The residue was dissolved in water and purified by filtration through a size-exclusion filter chosen to retain the product (29), estimated from of the size of the dendrimer used in the reaction. The product was several times washed with water, subsequently dissolved in water and freeze dried.

Compounds (30) and (31) were synthesized as described above using compounds (27) and (28) as starting materials.

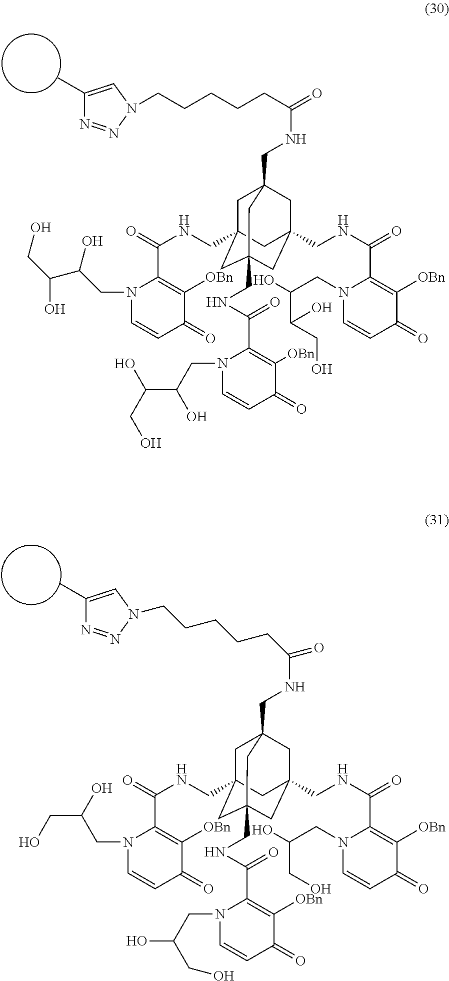

### Example 22

### Removal of Benzyl Protecting Group from Compound (29) to Give Compound (32)

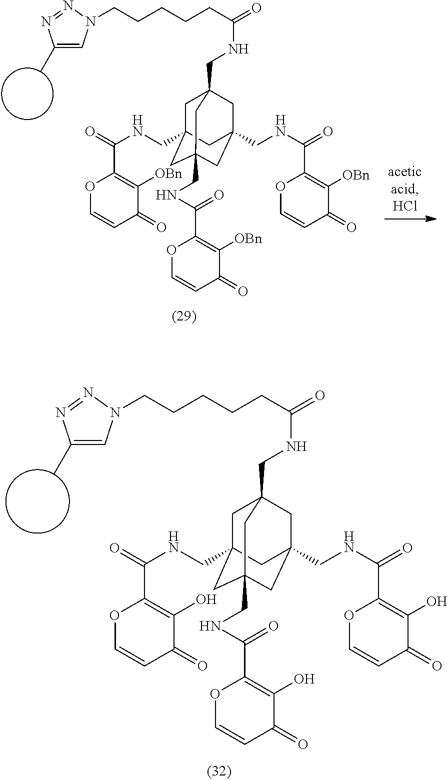

Compound (29) was dissolved in acetic acid and treated with an equal volume of concentrated hydrochloric acid. After 24 h the reaction was neutralised with an excess of a dilute aqueous ammonia solution and purified by filtration through a size-exclusion filter chosen to retain the product (32), estimated from of the size of the dendrimer. The product on the filter was several times washed with water and subsequently dissolved in water and freeze dried to give (32).

Compounds (33) and (34) were synthesized as described above using compounds (30) and (31) as starting materials.

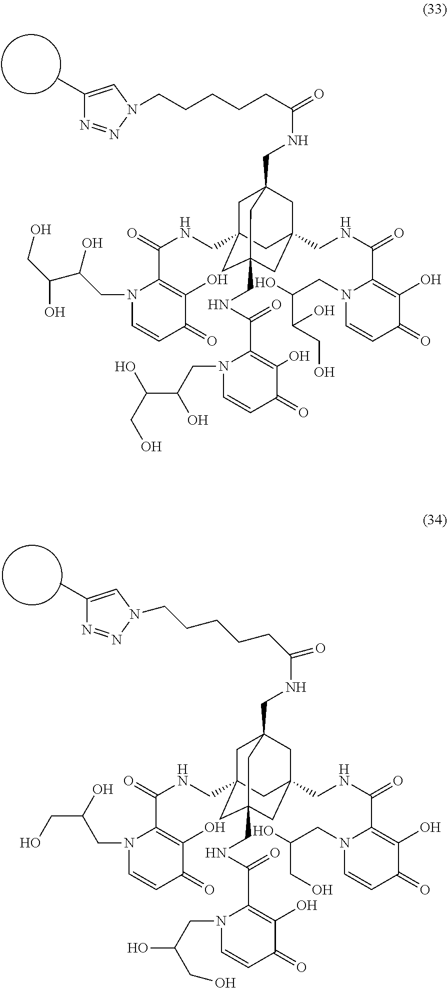

### Example 23

### Formation of a Paramagnetic Chelate (35) by Complexing Gd³⁺ with the Chelator (32)

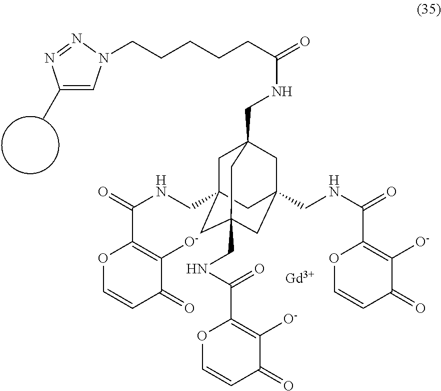

Compound (32) was dissolved in water and an excess of an aqueous Gd(III)acetate solution was added. The reaction mixture was stirred at room temperature for 2 h and then filtered through a size exclusion filter that retained the paramagnetic chelate (35). The paramagnetic chelate was several times washed with water, then dissolved in water and freeze dried.

Compounds (36) and (37) were synthesized as described above using compounds (33) and (34) as starting materials.

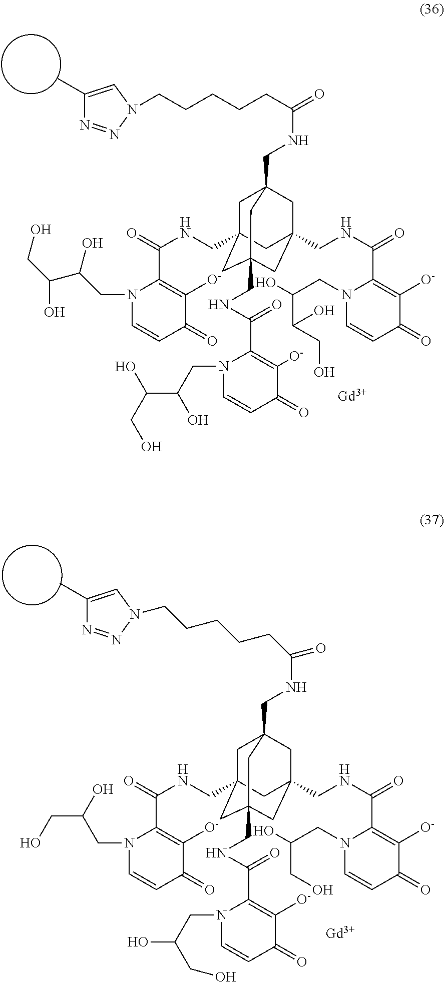

What is claimed is:

1. Compound of formula (I) 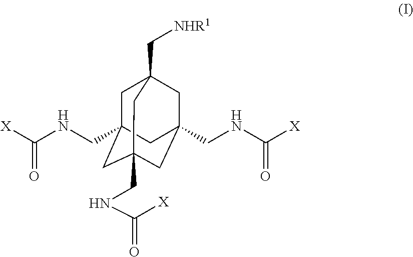
wherein
R¹ is H or C(═O)R², wherein R² is C₁₋C₆-alkyl, C₆₋C₁₅-aryl, C₇₋C₂₂-arylalkyl O—C₁₋C₆-alkyl, )—C₇₋C₂₂-arylalkyl, (CH₂)ₙ—(C₆H₄)—NCS, (CH₂)ₘ—C≡CH or (CH₂)ₘ—N₃, wherein n is 0 to 6 and m is 1 to 6; and
X is a chelator moiety consisting of a 6-membered aromatic or partially saturated ring system containing up to three heteroatoms selected from nitrogen and oxygen and having a hydroxyl group as a first substituent bound to a first atom in said ring system, and a hydroxyl group or an oxygen atom doubly bound to a second atom in said ring system wherein said first and second atom are adjacent atoms and wherein said first and second substituents are in ring positions such that X is capable of forming a complex with a paramagnetic metal ion; and wherein X is optionally substituted by up to three additional substituents, R, where each R is independently a hydrophilic group which renders the compound of formula (I) soluble in aqueous solutions.

2. Compound according to claim 1 further comprising a paramagnetic metal ion M to form a compound of formula (II): 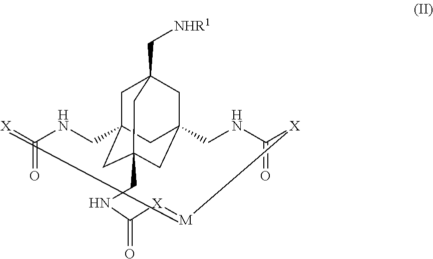
wherein
R¹ and X are as defined in claim 1 and M is a paramagnetic metal ion.

3. Compound according to claim 1 wherein X is derived from hydroxypyrones, dihydroxypyridines, hydroxypyrimidones, hydroxypyri-dones, hydroxy-pyridinones and dihydroxyphenols.

4. Compound according to claim 2 wherein M is a paramagnetic metal ion of Mn, Fe, La, Co, Ni, Eu, Gd, Dy, Tm and Yb.

5. Compound according to claim 1 wherein R¹ is H or C(═O)R², wherein R² is C₁₋C₆-alkyl, C₆₋C₁₅-aryl, C₇₋C₂₂-arylalkyl, O—C₁₋C₆-alkyl, O—C₇₋C₂₂-arylalkyl.

6. Compound according to claim 1 wherein R¹ is C(═O)R² wherein R² is (CH₂)ₙ—(C₆H₄)—NCS, (CH₂)ₘ—C≡CH or (CH₂)ₘ—N₃.

7. Compound according to claim 1 linked to another molecule via the NHR¹-group.

8. Compound according to claim 7 wherein said another molecule is a natural or synthetic peptide, a peptidomimetic, a polypeptide, a protein, an antibody, a natural or synthetic polymer, a dendrimer, a nanoparticle or a lipophilic compound.

9. Compound according to claim 7 wherein said another molecule comprises an amino group and is selected from a polypeptide, a protein, an antibody, a natural or synthetic polymer or a dendrimer and wherein said compound of formula (I) is linked to said another molecule via a thiourea bond formed by reaction of the NHR¹-group of compound of formula (I) wherein R¹ is C(═O)R² and R² is (CH₂)ₙ—(C₆H₄)—NCS and n is 0 to 6 with said amino groups of said another molecule.

10. Compound according to claim 7 wherein said another molecule comprises an azido group and is selected from a polypeptide, a protein, an antibody, a natural or synthetic polymer or a dendrimer and wherein said compound of formula (I) is linked to said another molecule via a 1,2,3-triazole ring formed by reaction of the NHR¹-group of compound of formula (I) wherein R¹ is C(═O)R² and R² is (CH₂)ₘ—C≡CH and m is 1 to 6 with said azido group of said another molecule.

11. Compound according to claim 7 or S wherein said another molecule comprises an ethynyl group and is selected from a polypeptide, a protein, an antibody, a natural or synthetic polymer or a dendrimer and wherein said compound of formula (I) is linked to said another molecule via a 1,2,3-triazole ring formed by reaction of the NHR¹-group of compound of formula (I) wherein R¹ is C(═O)R² and R² is (CH₂)ₘ—N₃ and m is 1 to 6 with said ethynyl group of said another molecule.

12. Compound according to claim 2 for use as MR contrast agents.

13. Composition comprising a compound according to claim 2 and at least one physiologically tolerable carrier.

14. Composition according to claim 13 for use as MR contrast medium in MRI.

15. Method of MR imaging wherein a composition according to claim 13 is administered to a subject and the subject is subjected to an MR examination wherein MR signals are detected from the subject or parts of the subject into which the composition distributes and optionally MR images and/or MR spectra are generated from the detected signals.

16. Method for producing the compound of formula (I) according to claim 1 wherein R¹ is H by
a) reacting a mono-protected 1,3,5,7-tetrakis(amino-methyl)adamantane with a compound of formula (IV)
X^{Z}—C(O)—Y (IV)
wherein
X^{Z} is X as defined in claim 1 and wherein the hydroxyl groups which are bound to X are protected;
Y is a leaving group; and
b) removing the amino protecting group of said mono-protected 1,3,5,7-tetrakis(aminomethyl)adamantane and the hydroxyl protecting groups of X^{Z}.

17. Method for producing the compound of formula (I) according to claim 1 wherein R¹ is C(═O)R² and R² is C₁₋C₆-alkyl, C₆₋C₁₅-aryl, C₇₋C₂₂-arylalkyl, O—C₁₋C₆-alkyl, O—C₇₋C₂₂-arylalkyl, (CH₂)ₘ—C≡CH or (CH₂)ₘ—N₃ and m is 1 to 6 by
a) reacting a mono-protected 1,3,5,7-tetrakis(aminomethyl)-adamantane with a compound of formula (IV)
X^{Z}—C(O)—Y (IV)
wherein
X^{Z} is X as defined in claim 1 and wherein the hydroxyl groups which are bound to X are protected;
Y is a leaving group;
b) removing the amino protecting group of said mono-protected 1,3,5,7-tetrakis-(aminomethyl)adamantane;
c) reacting the product obtained with a compound of formula (VI)
Y—C(═O)R² (VI); and
d) removing the hydroxyl protecting groups of X^{Z}.

18. Method for producing the compound of formula (I) according to claim 1 wherein R¹ is C(═O)R² and R² is (CH₂)ₙ—(C₆H₄)—NCS and n is 0 to 6 by
a) reacting a mono-protected 1,3,5,7-tetrakis(aminomethyl)-adamantane with a compound of formula (IV)
X^{Z}—C(O)—Y (IV)
wherein
X^{Z} is X as defined in claim 1 and wherein the hydroxyl groups which are bound to X are protected;
Y is a leaving group;
b) removing the amino protecting group of said mono-protected 1,3,5,7-tetrakis-(aminomethyl)adamantane;
c) reacting the product obtained with a compound of formula (VI_{A}*)
Y—C(═O)—(CH₂)ₙ—(C₆H₄)—NO₂ (VI_{A}*)
wherein n and Y are as defined above;
d) reducing the nitro group to an amino group;
e) reacting the amino group with thiophosgene to give the isothiocyanate group —N═C═S; and
removing the hydroxyl protecting groups of X^{Z}.

19. Method for producing a compound of formula (II) according to claim 2 by reacting a compound of formula (I) 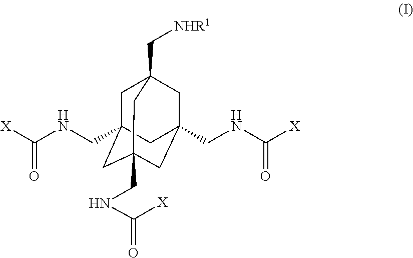
wherein
R¹ is H or C(═O)R², wherein R² is C₁₋C₆-alkyl, C₆₋C₁₅-aryl, C₇₋C₂₂-arylalkyl O—C₁₋C₆-alkyl, O—C₇₋C₂₂-arylalkyl, (CH₂)ₙ—(C₆H₄)—NCS, (CH₂)ₘ—C≡CH or (CH₂)ₘ—N₃, wherein n is 0 to 6 and m is 1 to 6; and
X is a chelator moiety consisting of a 6-membered aromatic or partially saturated ring system containing up to three heteroatoms selected from nitrogen and oxygen and having a hydroxyl group as a first substituent bound to a first atom in said ring system, and a hydroxyl group or an oxygen atom doubly bound to a second atom in said ring system wherein said first and second atom are adjacent atoms and wherein said first and second substituents are in ring positions such that X is capable of forming a complex with a paramagnetic metal ion; and wherein X is optionally substituted by up to three additional substituents, R, where each R is independently a hydrophilic group which renders the compound of formula (I) soluble in aqueous solutions with a paramagnetic metal ion.
